(19) United States
(12) Patent Application Publication
(10) Pub. No.: US 2013/0231725 A1
(43) Pub. Date: Sep. 5, 2013

(54) Peripheral Afferent Nerve Stimulation for Treatment of Epilepsy

(71) Applicants: Michael Williams, Santa Rosa, CA (US); Candace Borders, Santa Rosa, CA (US)

(72) Inventors: Michael Williams, Santa Rosa, CA (US); Candace Borders, Santa Rosa, CA (US)

(21) Appl. No.: 13/600,213

(22) Filed: Aug. 30, 2012

Related U.S. Application Data
(60) Provisional application No. 61/529,063, filed on Aug. 30, 2011.

(51) Int. Cl. A61N 1/05

(52) U.S. Cl. CPC A61N 1/0551
USPC 607/116

(57) ABSTRACT

A neuromodulation system for treating epilepsy provides therapeutic elements for modulating nerve activity to prevent or diminish (e.g., through reduced intensity or shortened duration) epileptic seizures. The therapeutic elements may be positioned in the vasculature of the patient or on the skin of the patient and are energized to modulate nerve fibers positioned outside the vascular walls. Electrode positions may include the surface of the hand and/or arm, or blood vessels within the hand and/or arm such as the vena comitans of the ulnar artery, the vena comitans of the radial arteries, the ulnar artery, or the radial artery. Alternative electrode positions are beneath the skin of the hand or arm but external to the blood vessels. Target nerves include the median nerve, radial nerve, ulnar nerve and/or branches thereof.

This application claims the benefit of U.S. Provisional Application No. 61/529,063, filed Aug. 30, 2011, which is incorporated herein by reference.

### FIELD OF THE INVENTION

The present invention relates generally to the field of nerve stimulation for treatment of epilepsy. More particularly, the invention relates to devices for treating epilepsy using electrical stimulation of afferent nerves in the hand and arm to reduce the frequency and severity of seizures.

### BACKGROUND

Epileptic seizures can have any of multiple and varied causes, and some of these causes are unknown. Generally, however, they can be characterized as abnormal electrical activity of neurons in the brain. These neurons are thought to become hyperexcitable and hypersynchronous. When electrical impulses are delivered to nerves and transmitted to the brain as action potentials, they serve to desynchronize the hypersynchronous activity.

Stimulation of the vagus and trigeminal nerves has been shown to decrease epileptic seizures in duration and frequency. The vagus nerve is a mixed. nerve (afferent and efferent fibers) innervating much of the viscera, and the trigeminal nerve is the primarily sensory afferent nerve innervating the mouth and face.

The efficacy of trigeminal nerve stimulation in reducing seizure activity is hypothesized to be partly attributable to the relatively large area of brain tissue corresponding to it the larger the area affected by neurostimulation, the more potential interference with abnormal, epileptic activity in the brain,

The size of the area of the cerebral cortex of the brain receiving incoming sensory data from any given part of the body is not necessarily proportional to the size of the body part itself This concept is best rendered by the “sensory homunculus,” or illustration of the human body with proportions adjusted to reflect the amount of brain area devoted to incoming sensory data from each anatomical structure. The homunculus illustrates the fact that, proportionally, the brain devotes a great deal of surface area to sensory input from the hands, as well as the tongue, lips and lower part of the face (areas innervated by the trigeminal nerve).

### BRIEF DESCRIPTION OF THE DRAWINGS:

FIG. 1 illustrates the sensory anatomy of the hand.

FIGS. 2-4 illustrate blood vessels and veins of the arms.

FIG. 5 schematically illustrates a neurostimulation system for treatment of epilepsy.

### DETAILED DESCRIPTION

Because trigeminal nerve stimulation has been shown to reduce epileptic seizure behavior, the present inventors believe that stimulation of the sensory afferent nerves innervating the fingers and hands will also produce such an effect, as a large portion of cortical space receives sensory input from these anatomical structures, The trigeminal nerve is functionally equivalent to a sensory nerve of the peripheral nervous system, and the two take similar paths to the somatosensory area of the cerebral cortex via the thalamus (thalmo-cortical pathway).

Therapeutic neuromodulation could be affected with either a surgically- or transvenously-delivered and implanted therapeutic element. The arm or hand as target locations for placement of a neurostimulation device have some preferable characteristics including but not limited to ease of access, low risk of side effects or complications, cosmesis and two regions of availability.

A device to perform this therapy uses a therapeutic element 10 which might be a pair or an array of electrodes or other source of energy (e.g. optical, acoustic, thermal etc.), and a pulse generator 12 in wired or wireless communication with the therapeutic element. See FIG. 5. The therapeutic element 10 is positioned to stimulate afferent nerves in the arm or hand. In one embodiment, the therapeutic element 10 may be delivered transvenously via a catheter or other low profile wire based system. In such cases, therapeutic energy is directed from the therapeutic element to the target nerve(s) external to the blood vessel in which the electrode or other therapeutic element is positioned. A pulse generator and or power supply 12 may be implanted in the arm, in the vasculature such as in the subclavian vein, or subcutaneously in the arm or chest, or it may be positioned outside. the body for wireless communication with the therapeutic element. Intravascular electrodes for use in transvascular stimulation of nerves are known in the art. See, for example, US Publication No. 2007/0255379, which is incorporated herein by reference and which also describes intravascular pulse generators.

In an alternative approach, the therapeutic element 10 is carried by a cutaneous patch for transcutaneous delivery of therapeutic stimulation to the target nerve(s). The patch is positioned on the hand or arm in position to deliver therapeutic stimulus through the skin to the target nerves. in this embodiment, the pulse generator 12 may be integrated with the same patch or a second patch, or the pulse generator may be a subcutaneous implant, or it may be an extracorporeal device in wireless communication with the therapeutic element, In another embodiment, the therapeutic element 10 is a subcutaneous therapeutic element (e.g. electrode array) delivered via a small incision or needle stick accompanied by a pulse generator 12. This embodiment may utilize a like form factor pulse generator, or a secondary patch, or a subcutaneous implant or other extracorporeal device in wireless communication with the therapeutic element.

Stimulation may be delivered to one or more of various possible target nerves in the hands and arms. The following discussion provides examples of such nerve targets suitable for receiving therapeutic stimulus from transcutaneous, transvascular, or subcutaneous therapeutic elements, and also discloses vessels within which the therapeutic element may be anchored in embodiments for transvascularly stimulating nerve targets.

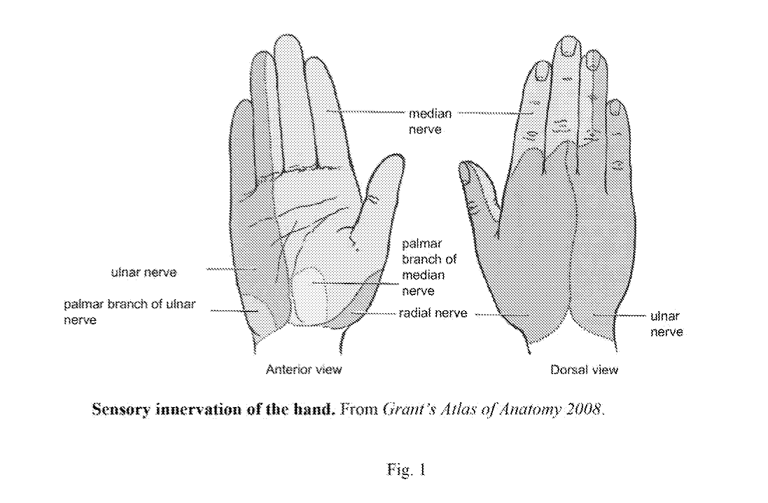

Three main nerves innervate the forearm and hand: the radial, ulnar, and median nerves. Each is mostly a mixed nerve (comprised of both sensory afferent and motor efferent neurons) with some areas dividing into separate sensory and motor branches. The median nerve, which innervates the palmar aspect of the thumb, index, and middle fingers, separates into sensory and motor branches at the wrist, while the ulnar nerve, which innervates the ring finger and little finger, divides proximal to the wrist. The radial nerve innervates the dorsal aspect of the thumb, index, and middle fingers. Except for one branch of the radial nerve, none of these innervate the upper arm. See FIG. 1.

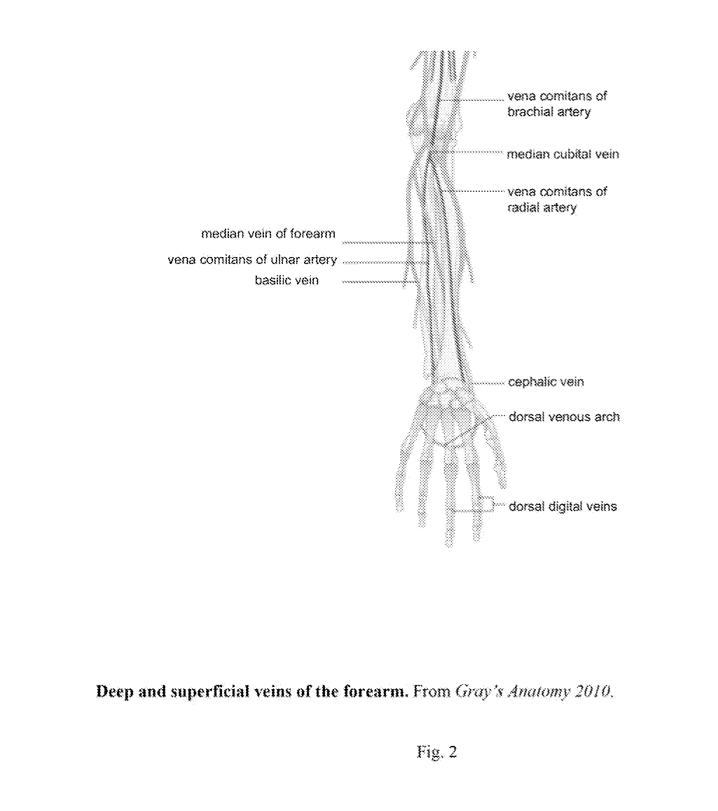

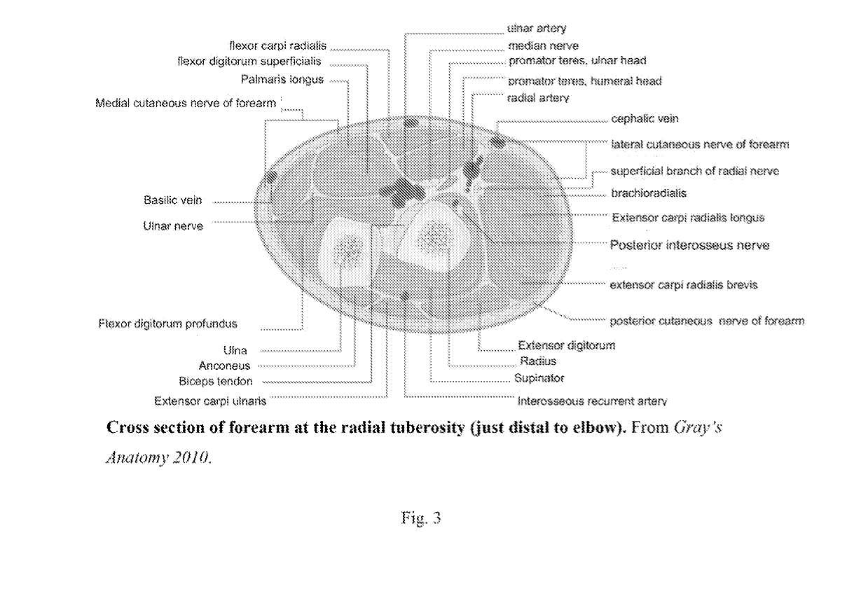

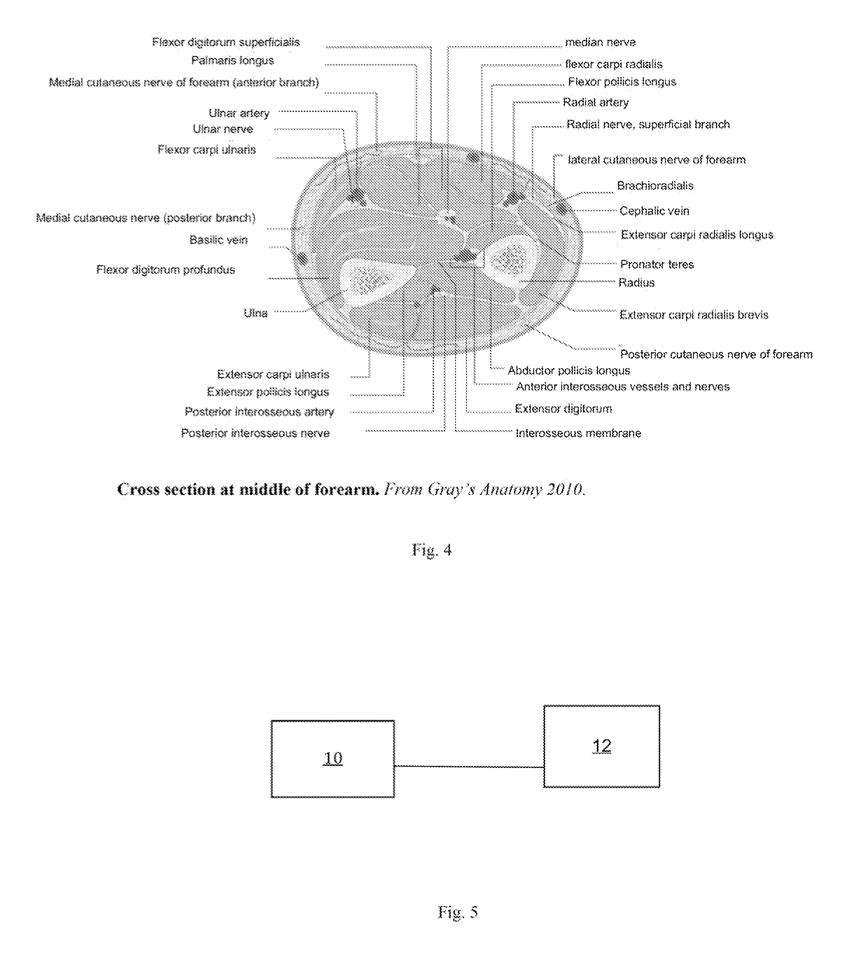

The forearms contain both deep and superficial veins that may provide vascular access to the relevant neuroanatomy for stimulation. The proximity of the median, radial, and ulnar nerve to specific vessels varies along the length of the arm but at certain points they are directly adjacent. Deep veins in the forearm include the vena comitantes of the radial and ulnar arteries. Superficial veins include the dorsal venous arch in the hand, and the cephalic, basilic, and median veins in the forearm. See FIGS. 2-4.

Just distal to the elbow, the median nerve is estimated to traverse the space adjacent to the vena comitans of the ulnar and radial arteries; either of these veins would likely provide an ideal placement for an intravascular therapeutic element capable of directing therapeutic energy to the median nerve. Closer to the hand, the ulnar nerve's proximity to the ulnar artery would also well serve this purpose—allowing a therapeutic element to be positioned in the ulnar artery for delivering therapeutic stimulus to the ulnar nerve. As another example, regions of the radial artery proximate to the radial nerve may be suitable implantation sites for a therapeutic element used to stimulate the radial nerve, such sites include those near the elbow and near the hand as well as regions intermediate of those sites.

Vagus and trigeminal nerve stimulation used therapeutically as a treatment for epileptic patients have been shown to reduce the frequency and severity of seizures with both chronic stimulation (to prevent seizures from occurring) or seizure-induced stimulation (to stop a seizure once it has begun). Therapeutic stimulation of the afferent nerves of the hand and arm could offer this same benefit, while eliminating the need to access the vasculature and nerves of the face.

This type of therapeutic stimulation has the potential to disrupt many of the types of seizures collectively described as epilepsy. Generalized seizures, which involve abnormal electrical signals throughout the brain, could be interfered with. Moreover, partial (or localized) seizures taking place in parts of the cerebral cortex near the somatosensory cortex could be treated. It is also possible for partial seizures to move or spread across the brain, as well as between the cortical and sub-cortical layers of the brain Thus, a. partial seizure with a point of origin other than the somatosensory cortex may still potentially be treated by sensory afferent neuromodulation.

The therapeutic element may be part of a system that senses physiological activity and determines the onset of an epileptic seizure or other changes indicative that a seizure is likely, and that delivers the neuromodulation therapy in response to the detected onset or change. in other embodiments, the patient may have an external controller in wireless communication with the therapeutic implant, allowing the patient to initiate neuromodulation when s/be senses the onset of an epileptic seizure.

We claim:

1. A method for treating epilepsy, the method comprising:
positioning a therapeutic element in proximity to at least one target afferent nerve of a hand and/or arm of a patient; and
delivering therapeutic energy from the therapeutic element to the target nerve such that delivery of the therapeutic energy prevents or diminishes epileptic seizure activity of the patient.

2. The method of claim 1, wherein the positioning step includes positioning the therapeutic element in a blood vessel of a hand and/or arm of a patient.

3. The method of claim 1, wherein the delivering step includes transcutaneously delivering the therapeutic energy through the skin of the hand and/or arm.

4. The method of claim 1, wherein the target nerve is selected from the group consisting of the radial nerve, ulnar nerve, and median nerves.

5. The method of claim 1, wherein the blood vessel is the vena comitans of the ulnar artery, the vena comitans of the radial arteries, the ulnar artery, or the radial artery.

6. A system for treating epilepsy, comprising:
a therapy element adapted for positioning within a blood vessel on a hand and/or arm of a patient; and
a stimulator configured to energize the therapy element within the blood vessel to deliver therapy to a nerve fiber disposed external to the blood vessel such that delivery of the therapy prevents or diminishes epileptic seizure activity of the patient.

7. A system for treating epilepsy, comprising:
a therapy element adapted for positioning within on a hand and/or arm of a patient, and
a stimulator configured to energize the therapy element to transcutaneously deliver therapy to a peripheral afferent nerve fiber within the hand and/or arm that delivery of the therapy prevents or diminishes epileptic seizure activity of the patient.
